(19) United States
(12) Patent Application Publication
(10) Pub. No.: US 2004/0215314 A1
(43) Pub. Date: Oct. 28, 2004

(54) Stent deployment assembly with collars for drug-eluting stent

(76) Inventors: John D. Kantor, Santa Rosa, CA (US); Ryan A. Jones, Santa Rosa, CA (US)

Correspondence Address: FRANK C. NICHOLAS, CARDINAL LAW GROUP, Suite 2000, 1603 Orrington Avenue, Evanston, IL, 60201, US

(21) Appl. No.: 10/827,819

(22) Filed: Apr. 20, 2004

Related U.S. Application Data
(60) Provisional application No. 60/465,496, filed on Apr. 25, 2003.

(51) Int. Cl. A61F 2/06

(52) U.S. Cl. 623/1.11; 623/1.42

(57) ABSTRACT

The present invention provides a system for treating a vascular condition, including a catheter and a stent deployment assembly coupled to the catheter. The stent deployment assembly includes a balloon, a first inelastic collar attached at a proximal end of the balloon, and a second inelastic collar attached at a distal end of the balloon. The collars limit expansion of the balloon at the proximal and distal ends of the balloon when the balloon is inflated.

### RELATED APPLICATION

This application claims priority to U.S. Provisional Application No. 60/465,496, “Stent Deployment Assembly with Collars for Drug-eluting Stent” to John D. Kantor and Ryan A. Jones, filed Apr. 25, 2003, the entirety of which is incorporated by reference.

### FIELD OF THE INVENTION

This invention relates generally to catheter deployment of drug-coated stents. More specifically, the invention relates to a stent deployment system and method that prevents the expansion of a catheter balloon into regions of a body vessel beyond the ends of the stent.

### BACKGROUND OF THE INVENTION

Stent deployment systems typically expand a stent into the luminal surface of a bodily vessel by using a balloon catheter. A small, expandable stent is slipped over a catheter and placed at the site where a vessel needs to be widened, reinforced, and prevented from narrowing again. An inflatable device such as a balloon is used to expand the stent to the desired size. In these systems, various approaches have been used to retain the stent on the catheter balloon and to control deployment until the stent is expanded into place.

The shape of the balloon may be used to help secure and position a stent. Bleam describes a catheter balloon with a tapered end in “Catheter Having Optimized Balloon Taper Angle”, U.S. Pat. No. 5,797,878 issued Aug. 25,1998. The angle between the tapered port and the catheter tube is selected to readily slide the collapsed balloon out of the vessel.

A specialized configuration of balloon folds has been used for retaining a stent until deployment. For example, a catheter balloon with a circumferential fold is used to retain the stent in place until balloon dilation, as disclosed in “Stent Securement by Balloon Modification”, Pederson et al., U.S. Pat. No. 6,280,412 issued Aug. 28, 2001. Daniel Adams describes another folded catheter balloon design in “Balloon with Reversed Cones”, U.S. Pat. No. 6,221,042 issued Apr. 24, 2001. The inflatable area of the balloon extends longitudinally over affixed ends of the balloon, and when folded or deflated, the portions of the balloon extending over the affixed ends provide a bulky mass onto which a stent may be securely crimped.

Some stent deployments systems use additional structures for securing a stent to a catheter balloon. Elastic sleeves may be used to fix a stent to a balloon catheter, as described in “Stent Delivery System”, U.S. Pat. No. 4,950,227, Savin et al., issued Aug. 21, 1990. These elastic silicone caps or sleeves are located over the ends of an expandable stent on a catheter balloon. As the balloon and stent are expanded, the caps slip off the ends of the stent and the stent is released. Use of one or more stent retaining sleeves are employed by Wang and others, as described in “End Sleeve Coating for Stent Delivery”, U.S. Pat. No. 6,443,980 issued Sep. 3, 2002 and “End Sleeve Coating for Stent Delivery”, U.S. Pat. No. 6,331,186 issued Dec. 18, 2001. The retaining sleeves, which cover the ends of a stent and the axially adjacent areas of the balloon, may have a lubricious coating on its inside and outside surfaces, also described in “Lubricated Sleeve Material for Stent Delivery”, Wang et al., U.S. Pat. No. 6,221,097 issued Apr. 24, 2001.

A stent deployment system may use a combination of an annular collar and at least one cup to secure a stent onto the catheter balloon, as disclosed by Ellis et al. in “Stent Delivery Device Using Stent Cups and Mounting Collars”, U.S. Pat. No. 6,395,008 issued May 28, 2002. The collar and cup are coaxially located on the distal end of a catheter. The catheter balloon is inflated to expand the stent and release the stent from one or more cups, which can be axially spaced from the collar.

While these stent deployment systems have been successful in securing the stent to the catheter, the systems are normally unable to have a perfect correspondence between the length of the balloon and the length of the stent. An insufficiently long balloon fails to satisfactorily enlarge the stent at one or both ends, increasing the risk of thrombosis and end-stent restenosis. Typically there is too much balloon material, and thus, at least a portion of a vessel at each end of the catheter balloon is expanded by the balloon without the presence of a stent thereafter. This uncontrolled expansion beyond the ends of the stent may create undesired effects within the vessel, increasing the risk of end-stent restenosis. Recent studies on drug-coat stents have found a higher degree of restenosis in the regions of the vessel next to the ends of the stent than the area of the vessel corresponding to the middle of the stent. Release of therapeutic compounds from the drug-coated stent may help in the prevention and healing of tissue damage where the stent is deployed, though the released drugs may have reduced or little effect on damaged tissue upstream or downstream from the deployed stent. Undue expansion of a vessel without a drug-coated stent deployed thereafter ultimately may create undesired effects within to the vessel.

Therefore, it is desirable to have a stent deployment system and method with greater control of the balloon expansion and size, a balloon with the precise length of the stent to be deployed, and a way to prevent the expansion of the balloon into regions of the vessel beyond the stent ends. In addition, a desirable system and method would increase the effectiveness of drug-coated stents and lessen in-stent and end-stent restenosis.

### SUMMARY OF THE INVENTION

One aspect of the invention provides a system for treating a vascular condition including a catheter and a stent deployment assembly. The stent deployment assembly includes a balloon, a first inelastic collar attached at a proximal end of the balloon, and a second inelastic collar attached at a distal end of the balloon. The collars limit expansion of the balloon at the proximal and distal ends of the balloon when the balloon is inflated.

Another aspect of the invention is a method of deploying a drug-polymer coated stent in a vessel. A drug-polymer coated stent is positioned in the vessel with a catheter coupled to the drug-polymer coated stent. A balloon coupled to the drug-polymer coated stent inflates. Expansion of a proximal end and a distal end of the balloon is limited with a first inelastic collar attached to the proximal end of balloon and a second inelastic collar attached to the distal end of the balloon. Extension of the balloon beyond a proximal end and a distal end of the drug-polymer coated stent is prevented when the balloon is inflated. The balloon collapses, deploying the drug-polymer coated stent in the vessel.

Another aspect of the invention provides a method of manufacturing a stent deployment assembly. A balloon is coupled to a catheter. A first inelastic collar is fixedly attached at a proximal end of the balloon. A coated stent including a stent framework and a drug-polymer coating disposed on the stent framework is positioned proximate the balloon and the first inelastic collar. The coated stent compresses onto the balloon, such that a proximal end of the stent framework is compressed adjacent to the first inelastic collar.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

FIG. 1 is an illustration of a system for treating a vascular condition including a catheter and a stent deployment assembly, in accordance with one embodiment of the current invention;

FIG. 2 is an illustration of a stent coupled to a balloon;

FIG. 3 is an illustration of a stent deployment assembly including a coated stent, a balloon, and a set of collars, in accordance with one embodiment of the current invention;

FIG. 4 is a flow diagram of a method for deploying a drug-polymer coated stent in a vessel, in accordance with one embodiment of the current invention; and

FIG. 5 is a flow diagram of a method of manufacturing a stent deployment assembly, in accordance with one embodiment of the current invention.

### DETAILED DESCRIPTION OF THE PRESENTLY PREFERRED EMBODIMENTS

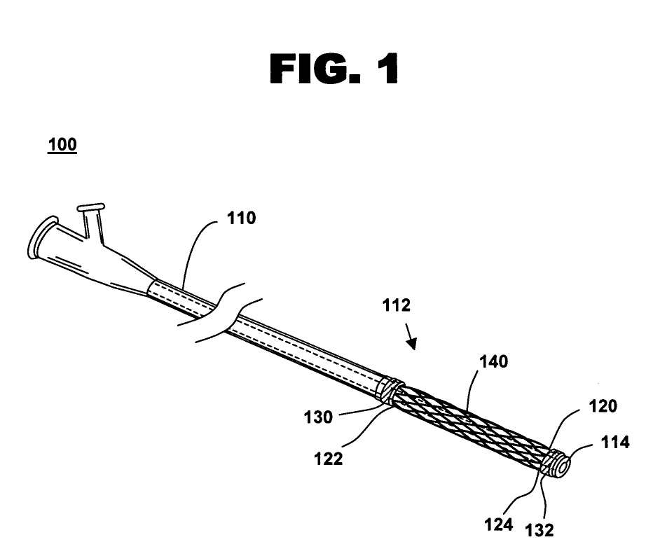

FIG. 1 shows an illustration of a system for treating a vascular condition, in accordance with one embodiment of the present invention at 100. Vascular condition treatment system 100 includes a catheter 10 and a stent deployment assembly 112 coupled to catheter 110. Stent deployment assembly 112 includes a catheter balloon 120, a set of collars 130 and 132, and a coated stent 140.

A first substantially rigid or inelastic collar 130 is attached at a proximal end 122 of balloon 120. A second inelastic collar 132 is attached at a distal end 124 of balloon 120. Collars 130 and 132 limit expansion of balloon 120 at proximal end 122 and distal end 124 when balloon 120 is inflated and coated stent 140 is being deployed.

Collars 130 and 132 are fixedly attached to catheter 110, holding balloon 120 firmly in place near each end of coated stent 140 against catheter 110. The working length of the balloon corresponds to the length of coated stent 140 being deployed. The working length is limited and controlled through the use of a pair of metal rings, fixed bands or collars 130 and 132 positioned around the outer periphery of balloon 120. Collars 130 and 132 function as a girdle to prevent expansion of balloon 120 in regions where there is no stent. Collars 130 and 132 limit balloon taper rollout or axial extension of balloon 120 and limit radial expansion of balloon 120 at proximal end 122 and distal end 124.

One or more therapeutic agents are in contact with polymeric coatings on coated stent 140. Vascular condition treatment system 100 may help treat, for example, heart disease, cardiovascular ailments, and other vascular conditions by using catheter-deployed endovascular stents that have tailored polymeric coatings for controlling the timed-release properties of interdispersed or encased therapeutic agents. Treatment of vascular conditions may include the prevention or correction of various ailments and deficiencies associated with the cardiovascular system, the cerebrovascular system, urinogenital systems, biliary conduits, abdominal passageways and other biological vessels within the body.

When assembled, an inner surface of coated stent 140 is positioned adjacent to balloon 120. In one embodiment, coated stent 140 is constrained from axial movement by collars 130 and 132, retaining coated stent 140 securely between collars 130 and 132 during shipping, handling, and positioning in the body. During deployment, balloon 120 inflates and presses outwardly against coated stent 140, enlarging coated stent 140 and deploying coated stent 140 within a vessel of a body.

To inflate balloon 120, a liquid such as a saline solution or a contrast fluid containing radiopaque material is injected into a lumen within catheter 110 and into the interior of balloon 120. The liquid is pressurized through a port in catheter 110 with an external controller, inflating balloon 120 and coated stent 140 until the desired diameter of coated stent 140 is obtained. Coated stent 140 generally retains its enlarged shape, pressing outwards against the vessel wall and being secured in part by the tissue bed and vascular wall surrounding coated stent 140. The pressure applied to the liquid is reduced and balloon 120 collapses. After successful deployment and implantation of coated stent 140, catheter 110 and balloon 120 are typically retracted into a guide catheter and removed from the body. An inner member 114 is typically included inside catheter 110 for a guide wire used to position catheter 110 in the body. The guide wire is inserted through another port in catheter 110.

Although depicted with a drug-polymer coated stent, stent deployment assembly 112 may comprise a balloon 120 with collars 130 and 132, a catheter 110, and a bare or uncoated stent, a drug-coated stent, a polymer-coated stent, or no stent at all. An embodiment with no stent at all may be used, for example, for pre-dilatation and post-dilatation maneuvers within a vessel. Expansions and extensions of proximal end 122 and distal end 124 of balloon 120 are restricted with collars 130 and 132 when balloon 120 is inflated.

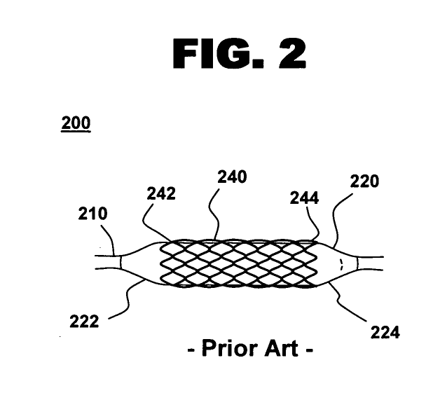

FIG. 2 shows an illustration of a stent coupled to a balloon, as generally known in prior art. With this stent deployment system 200, stent 240 is positioned over a balloon 220 that is adhered at each end to a catheter 210. Margin regions at a proximal end 222 and a distal end 224 of balloon 220 extend beyond a proximal end 242 and distal end 244 of stent 240. The margin regions allows balloon 220 to be expanded throughout its length, while the margins regions generally expand unencumbered by stent 240 and may cause undue expansion beyond the ends of stent 240 to a vessel in which stent 240 is being deployed. The regions beyond the ends of stent 240 may be difficult to treat with drugs and other therapeutic agents eluted from coatings on the stent.

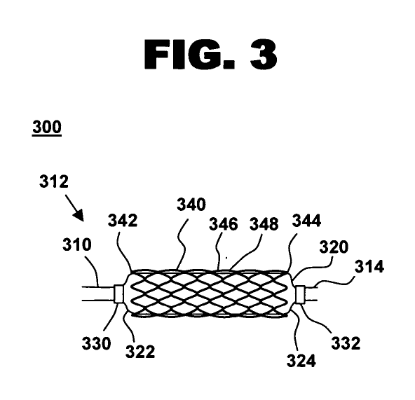

FIG. 3 shows an illustration of a stent deployment assembly including a coated stent, a balloon, and a set of collars, in accordance with one embodiment of the present invention at 300. Stent deployment assembly 300 includes a catheter 310 and a stent deployment assembly 312 coupled to the catheter. The stent deployment assembly 312 includes a balloon 320, a set of collars 330 and 332, and a coated stent 340.

A first inelastic collar 330 is attached to catheter 310 at a proximal end 322 of balloon 320. A second inelastic collar 332 is attached to catheter 310 at a distal end 324 of balloon 320. The collars 330 and 332 limit expansion at the proximal end 322 and distal end 324 of balloon 320 when balloon 320 is inflated. The margin regions at each end of balloon 320 extending beyond a proximal end 342 and a distal end 344 of coated stent 340 are reduced or eliminated by collars 330 and 332. Collars 330 and 332 are positioned adjacent to a proximal end and a distal end of coated stent 340. Collars 330 and 332 may abut the ends of coated stent 340 to limit expansion of proximal end 322 and distal end 324 of balloon 320 when balloon 320 is inflated. Collars 330 and 332 may abut or be placed a small distance from the ends of coated stent 340 to prevent the extension of balloon 320 beyond proximal end 342 and distal end 344 of coated stent 340.

Collars 330 and 332 may comprise a relatively inelastic material such as stainless steel, nitinol, tantalum, MP35N alloy, platinum, titanium, a thermoset plastic, a suitable biocompatible alloy, a suitable biocompatible material, or a combination thereof. Collars 330 and 332 may be made from or include a radiopaque marker comprising material such as platinum, barium, iridium, gold or a combination thereof. Collars 330 and 332 are typically three to thirty thousandths of an inch thick with a length typically between 0.1 millimeters and three millimeters or longer. In one embodiment, collar 330 is positioned adjacent to proximal end 342 of stent framework 346 and collar 332 is positioned adjacent to distal end 344 of stent framework 346. A drug-polymer coating 348 may be disposed on stent framework 346.

Coated stent 340 includes a stent framework 346 and a drug-polymer coating 348 disposed on at least a portion of stent framework 346. Stent framework 346 may comprise a polymeric base or a metallic base such as stainless steel, nitinol, tantalum, MP35N alloy, platinum, titanium, a suitable biocompatible alloy, a suitable biocompatible material, a suitable polymeric material, or a combination thereof. The polymeric base material may comprise any suitable polymer for biomedical stent applications, as is known in the art. Drug-polymer coating 348 may include or encapsulate one or more therapeutic agents.

Drug-polymer coating 348 may comprise one or more therapeutic agents dispersed within or encased by a polymeric coating, which are eluted from coated stent 340 with controlled time delivery after deployment of coated stent 340 within a body. A therapeutic agent is capable of producing a beneficial affect against one or more conditions including coronary restenosis, cardiovascular restenosis, angiographic restenosis, arteriosclerosis, hyperplasia, and other diseases and conditions. For example, the therapeutic agent can be selected to inhibit or prevent vascular restenosis, a condition corresponding to a narrowing or constriction of the diameter of the bodily lumen where the stent is placed. Drug-polymer coating 348 may comprise, for example, an antirestenotic drug, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, or combinations thereof. The elution rates of the therapeutic agents into the body and the tissue bed surrounding the stent framework are based on the constituency and thickness of drug-polymer coating 348, the nature and concentration of the therapeutic agents, the thickness and composition of any barrier or cap coats, and other factors.

Balloon 320 comprises an elastic material such as polyurethane, a thermoplastic polyamide such as Nylon™, low-density polyethylene (LDPE) polyethylene terephthalate (PET), or a thermoplastic elastomer, as is currently known in the art. Balloon 320 is generally cylindrical and elongated, and may include one or more folds or pleats. As balloon 320 inflates, balloon 320 expands against stent framework 346, causing plastic deformation of stent framework 346 to secure coated stent 340 within a vessel of a body. Inelastic collars 330 and 332 limit expansion of balloon 320 beyond proximal end 342 and distal end 344 of stent framework 346. As pressure is relieved, balloon 320 separates from coated stent 340. Balloon 320 is deflated into the original, compact shape about an inner member 314 of catheter 310. Coated stent 340 separates from balloon 320, and is retained at a directed location against a vascular wall while balloon 320 and catheter 310 are retracted and removed from the body.

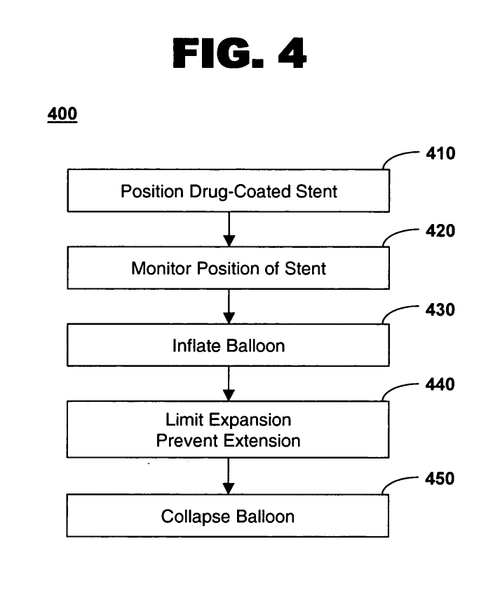

FIG. 4 shows a flow diagram of a method for deploying a drug-polymer coated stent in a vessel, in accordance with one embodiment of the present invention at 400. Coated stent deployment method 400 includes various steps to deploy a drug-polymer coated stent in a vessel of a body.

A drug-polymer coated stent is positioned in a vessel in the body, as seen at block 410. The drug-polymer coated stent is positioned in the vessel with a catheter coupled to the drug-polymer coated stent. The vessel may be located in one of many vessels within the cardiovascular system, or in other vascular systems within the body such as the cerebrovascular system, the urinogenital system, biliary conduits, abdominal passageways, or peripheral vasculature. A catheter coupled to the drug-polymer coated stent and a guide wire are inserted into one of the vessels of the body such as the femoral artery, and the coated stent is guided through one or more vessels into a directed or specified location within the body. The guide wire and catheter are manually manipulated through the vascular system to the desired location for stent deployment.

The position of the coated stent may be monitored, as seen at block 420. The coated stent position may be monitored, for example, using one or more radiopaque markers mounted on the stent, the stent deployment assembly or on the catheter. In one presently preferred embodiment, the radiopaque marker is located on at least one of the inelastic collars. In another presently preferred embodiment, radiopaque material is used in the construction of the inelastic collars. Alternatively, the position of an inflated or partially inflated balloon may be monitored with infilled radiopaque fluid or contrast fluid and associated x-ray imaging systems.

The catheter balloon is inflated, as seen at block 430. The balloon is coupled to the drug-polymer coated stent and is typically filled with a liquid, such as a contrast fluid that is fluidly coupled through the catheter from a source external to the body. As pressure is applied to the fluid, the balloon enlarges and the drug-polymer coated stent expands.

Expansion of the balloon is limited and extension of the balloon is prevented, as seen at block 440. The expansion of a proximal end of the balloon and a distal end of the balloon is limited with inelastic collars attached to the proximal end and the distal end of the balloon. The collars are fixedly attached to the catheter when the stent is being deployed. The collars prevent extension of the balloon beyond a proximal end and a distal end of the drug-polymer coated stent when the balloon inflates. The proximal end of the drug-polymer coated stent is positioned adjacent to the first inelastic collar, abutted against the collar or spaced a small distance from the collar. The distal end of the drug-polymer coated stent is positioned adjacent to the second inelastic collar, abutted against the collar or spaced a small distance from the collar.

The coated stent is deployed with the expanding balloon, enlarged and secured against the tissue bed of the vascular wall. The size of the deployed stent is determined in part by the maximum pressure applied to the fluid when inflating the balloon.

When the coated stent is suitably enlarged and secured in the vessel, the balloon is deflated and collapses, as seen at block 450. The pressure applied to the interior of the balloon is reduced and the coated stent separates from the balloon. Liquid in the balloon is pumped or vacuumed out, collapsing the balloon and deploying the coated stent. The balloon, collars and catheter are then withdrawn from the vessel.

After deployment of the coated stent (post-dilatation) or in the case of a balloon with collars and no stent affixed to a catheter (pre-dilatation), the balloon may be inflated within the vessel as desired to locally enlarge the vessels, as is typically done in balloon angioplasty and balloon dilatation procedures.

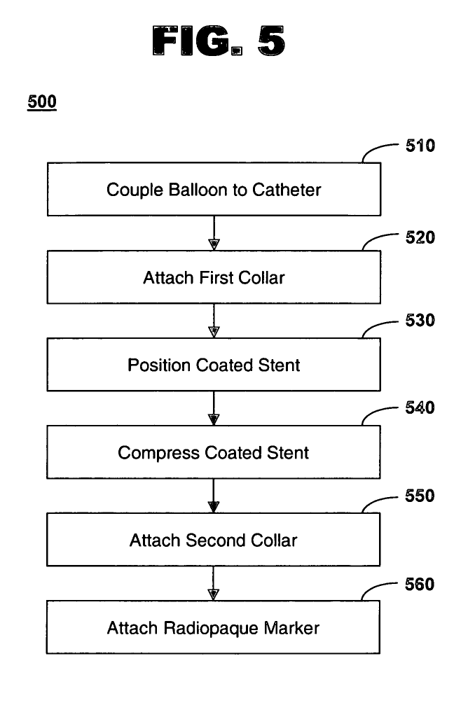

FIG. 5 shows a flow diagram of a method of manufacturing a stent deployment assembly, in accordance with one embodiment of the present invention at 500. Stent deployment assembly method 500 includes steps to manufacture a stent deployment assembly including a catheter, a balloon, at least one inelastic collar, and a coated stent with a stent framework and a drug-polymer coating on at least a portion of the stent framework.

A balloon is coupled to the catheter, as seen at block 510. A catheter balloon comprising polyurethane or other suitable elastic material, such as polyamide, low-density polyethylene (LDPE), polyethylene terephthalate (PET), or a thermoplastic elastomer, is typically coupled to the catheter at a distal end of the catheter. The catheter contains an inflation lumen for directing fluid into the balloon during deployment of the stent. The balloon overlaps at least one hole through the catheter wall for infilling the balloon with fluid. The elongated balloon is attached to the catheter at a proximal end of the balloon and a distal end of the balloon. The balloon is attached to the catheter using, for example, a heat bond. Heat bonds attach the balloon to the catheter using convection, conduction, laser, or ultrasonic heating techniques. One or more inelastic collars may be used to attach the balloon to the catheter with or without the use of heat bonds, glue, or epoxy.

A first inelastic collar is attached to the catheter, as seen at block 520. The first inelastic collar is fixedly attached to the catheter at a proximal end of the balloon, partially overlapping or adjacent to the heat bond. The first inelastic collar may be attached to the catheter using any suitable technique such as crimping, coining, or gluing.

A coated stent is positioned proximate the balloon and the first inelastic collar, as seen at block 530. The coated stent includes a stent framework and a drug-polymer coating disposed on the stent framework. The coated stent may be positioned such that a proximal end of the stent framework abuts or has a small gap next to the first inelastic collar. A fixture may be used to position the coated stent with the desired amount of margin next to the first inelastic collar.

The stent framework is compressed onto the balloon with the proximal end of the stent compressed adjacent to the first inelastic collar, as seen at block 540. The coated stent is compressed adjacent to the first inelastic collar to prevent extension of the balloon beyond a proximal end of the coated stent when deploying the stent. The drug-polymer coated stent is pressed against an outside surface of the balloon. The stent framework is compressed, for example, by rolling the coated stent down to the desired diameter in a roll-down machine. The coated stent may be rolled down to an outer diameter that equals, exceeds or is less than the outer diameter of the first inelastic collar.

A second inelastic collar is attached to the catheter, as seen at block 550. The second inelastic collar is fixedly attached to the catheter at a distal end of the balloon. The second inelastic collar is fixedly attached adjacent to a distal end of the stent framework, abutting against the stent framework or with a small space between the collar and the stent framework. The second inelastic collar is attached to the distal end of the balloon to prevent extension of the balloon beyond a distal end of the coated stent when deploying the coated stent. The second inelastic collar may be attached using crimping, coining, gluing, or other suitable technique. Alternatively, both inelastic collars may be attached to the catheter prior to positioning and compressing the coated stent. In one preferred embodiment, a collar at each end of the coated stent retains the compressed stent between the collars until the balloon is inflated to aid in stent retention during shipping, handling, and positioning in the body. In another embodiment, the second inelastic collar may be attached after a coated or uncoated stent is slid over the balloon, the stent not requiring compression.

A radiopaque marker may be attached to the catheter, as seen at block 560. The radiopaque marker may be attached, for example, to the first inelastic collar, the second inelastic collar, or both. In one presently preferred embodiment, the inelastic collars comprise a radiopaque material such as platinum, barium, iridium, gold, or a combination thereof. In another embodiment, the radiopaque marker is attached to the catheter prior to coupling the balloon.

The catheter with the stent deployment assembly and the coated stent is typically placed in a catheter package and sterilized prior to shipping and storing. Sterilization of the stent using conventional means is completed before clinical use.

Variations and alterations in the manufacture of the stent deployment assembly such as the specific order of collar, stent and radiopaque marker attachment are apparent to one skilled in the art, and may be made without departing from the spirit and scope of the present invention. While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A system for treating a vascular condition, comprising:
a catheter; and
a stent deployment assembly coupled to the catheter, the stent deployment assembly comprising a balloon, a first inelastic collar attached at a proximal end of the balloon, and a second inelastic collar attached at a distal end of the balloon; wherein the collars limit expansion of the balloon at the proximal end of the balloon and the distal end of the balloon when the balloon is inflated.

2. The system of claim 1 wherein the collars are fixedly attached to the catheter.

3. The system of claim 1 wherein the collars comprise a material selected from the group consisting of stainless steel, nitinol, tantalum, MP35N alloy, platinum, titanium, a thermoset plastic, a suitable biocompatible alloy, a suitable biocompatible material, and a combination thereof.

4. The system of claim 1 wherein the collars comprise a radiopaque marker.

5. The system of claim 1 wherein the stent deployment assembly further comprises:
a stent framework coupled to the balloon, the stent framework including a proximal end and a distal end, wherein the collars limit expansion of the balloon beyond the proximal and distal ends of the stent framework when the balloon is inflated.

6. The system of claim 5 wherein the proximal end of the stent framework is positioned adjacent to the first inelastic collar.

7. The system of 5 wherein the distal end of the stent framework is positioned adjacent to the second inelastic collar.

8. The system of claim 5 wherein the stent framework comprises one of a metallic base or a polymeric base.

9. The system of claim 5 wherein the metallic base comprises a material selected from the group consisting of stainless steel, nitinol, tantalum, MP35N alloy, platinum, titanium, a suitable biocompatible alloy, a suitable biocompatible material, and a combination thereof.

10. The system of claim 5 further comprising:
a drug-polymer coating disposed on the stent framework.

11. A method of deploying a drug-polymer coated stent in a vessel, the method comprising:
positioning the drug-polymer coated stent in the vessel with a catheter coupled to the drug-polymer coated stent;
inflating a balloon coupled to the drug-polymer coated stent;
limiting expansion of a proximal end and a distal end of the balloon with a first inelastic collar attached to the proximal end of the balloon and a second inelastic collar attached to the distal end of the balloon;
preventing extension of the balloon beyond a proximal end and a distal end of the drug-polymer coated stent when the balloon is inflated; and
collapsing the balloon to deploy the drug-polymer coated stent in the vessel.

12. The method of claim 11, wherein the collars are fixedly attached to the catheter when the stent is being deployed.

13. The method of claim 11 further comprising:
monitoring the position of the drug-polymer coated stent in the vessel with the collars, the collars comprising a radiopaque marker.

14. A method of manufacturing a stent deployment assembly, the method comprising:
coupling a balloon to a catheter;
fixedly attaching a first inelastic collar at a proximal end of the balloon;
positioning a coated stent proximate the balloon and the first inelastic collar, the coated stent including a stent framework and a drug-polymer coating disposed on the stent framework; and
compressing the coated stent onto the balloon, a proximal end of the stent framework compressed adjacent to the first inelastic collar.

15. The method of claim 14 wherein the coated stent is compressed adjacent to the first inelastic collar to prevent extension of the balloon beyond a proximal end of the coated stent when deploying the coated stent.

16. The method of claim 14 wherein the first inelastic collar is attached to the catheter by crimping, coining, or gluing.

17. The method of claim 14 further comprising:
fixedly attaching a second inelastic collar at a distal end of the balloon.

18. The method of claim 17 wherein the second inelastic collar is fixedly attached adjacent to a distal end of the stent framework.

19. The method of claim 17 wherein the second inelastic collar is attached to the distal end of the balloon to prevent extension of the balloon beyond a distal end of the coated stent when deploying the coated stent.

20. The method of claim 14 further comprising:
attaching a radiopaque marker to at least the first inelastic collar.
